(19) United States
(12) Patent Application Publication
(10) Pub. No.: US 2026/0237465 A1
(43) Pub. Date: Aug. 13, 2026

(54) A METHOD FOR DETERMINING A STAGE OF AN EPIGENETIC PROPERTY OF AN EPIDERMIS

(71) Applicant: MITRA BIO LIMITED, London (GB)

(72) Inventors: Cristiana BANILA, London (GB); Shakiba KAVEH, London (GB); Angel MENENDEZ VAZQUEZ, London (GB)

(21) Appl. No.: 19/160,622

(22) PCT Filed: Feb. 28, 2024
(86) PCT No.: PCT/EP2024/055144

§ 371 Date: Aug. 28, 2025

(30) Foreign Application Priority Data
Feb. 28, 2023 (GB) 2302959.8

(51) Int. Cl. G16B 40/20; C12Q 1/6876; C12Q 1/6886; G06N 20/10; G16B 30/00

(52) U.S. Cl. CPC G16B 40/20; C12Q 1/6876; C12Q 1/6886; G06N 20/10; G16B 30/00; C12Q 2600/112; C12Q 2600/154

(57) ABSTRACT

A method for determining a stage of an epigenetic property of an epidermis of a subject, comprising providing a keratinocyte sample from the subject, identifying methylated nucleic acid sequences in the keratinocyte sample by enzymatic methyl sequencing (EM-seq) and using a trained machine-learning model to process the identified methylated nucleic acid sequences and thereby to determine the stage of the epigenetic property.

### FIELD OF THE INVENTION

The invention comprises a method and a system for determining a stage of an epigenetic property of an epidermis of a subject, and a computer-implemented method for training a machine-learning model for determining the stage of the epigenetic property.

### BACKGROUND OF THE INVENTION

In the field of biotechnology and, in particular, in diagnostics, it is known to collect skin samples using invasive biopsies from a subject. This is the gold standard for deoxyribonucleic acid (DNA) methylation analysis and involves conducting bisulfite conversion on the collected skin samples to carry out subsequent analysis on either EPIC arrays, produced by Illumina, San Diego, California, USA, or using other sequencing platforms such as Illumina, Nanopore or PacBio technologies. High DNA levels are needed for preparing the skin samples for the bisulfite conversion. These high DNA levels in the skin samples were previously yielded only by invasive skin sampling.

Biopsies are one example of the invasive skin sampling. These biopsies require time and discomfort during the skin sampling. Multiple biopsies may be needed to track diagnostic results and obtain high DNA levels. Few subjects are willing to undergo the multiple biopsies because of the discomfort. Therefore, the invasive skin sampling is not a scalable method for DNA methylation analysis.

Other existing methods to obtain high DNA levels commonly rely on suction blister methods.

The suction blister methods, like the biopsies, cause discomfort to the subject and this discomfort discourages repeated sampling. The collection of the skin samples also necessitates in most cases a trained professional, i.e., clinician. This requirement of a trained professional prevents self-sampling and imposes logistical constraints on the sampling. The obtained skin collected by the trained professionals and the biopsies are often a mixture of dermis and epidermis in unknown proportions.

Sequencing of heterogenous cell mixtures of the mixture of dermis and epidermis using bulk omics' approaches has potential to be confounded by differing cell type proportions arising from cell-type specific DNA methylation profiles. Comparison to reference datasets can help deconvolute DNA methylation patterns and cell type in whole blood. Houseman, E. A., Accomando, W. P., Koestler, D. C. et al. “DNA methylation arrays as surrogate measures of cell mixture distribution,” BMC Bioinformatics 13, 86 (2012). https://doi.org/10.1186/1471-2105-13-86 have written that no reference datasets currently exist for the sampling of the skin.

Single-cell sequencing approaches may mitigate this issue of the heterogenous cell mixtures, but the single-cell sequencing approaches introduce other logistical challenges at scale.

U.S. Pat. No. 10,407,729 B2 (Dermtech) discloses a non-invasive method for diagnosing epidermal properties (including melanoma and/or solar lentigo) of a subject by analyzing ribonucleic acid (RNA) molecules obtained from a subject, such as a human being. The method comprises providing an epidermal sample from the subject by applying an adhesive tape on a skin area of the subject. This extraction of the epidermal sample is followed by identifying nucleic acid sequences in the epidermal sample by a microarray analysis or a direct sequencing method. The identification is followed by processing the identified nucleic acid sequences by performing an Ingenuity Pathways analysis and a Random Forests analysis on the nucleic acid sequences.

U.S. Pat. No. 7,297,480 B2 (Dermtech) discloses a further non-invasive method for detecting malignant melanoma in a skin sample of a subject. The method also comprises applying an adhesive surface on to the skin of the subject followed by removing the adhesive surface to obtain an epidermal sample of the subject. Ribonucleic acid sequences in the epidermal sample are identified by phosphor-imaging. The identified nucleic acids are, for example, endothelin-2, ephrin-A5 or Activin A. The method further comprises processing the identified nucleic acid sequences by analyzing the expression of the nucleic acid sequences with a non-parametric test.

U.S. Pat. No. 7,989,165 B2 (Dermtech) also discloses a non-invasive method for determining skin disease and pathological skin states, such as irritated skin and psoriasis. The method comprises providing the epidermal sample from the subject by application of an adhesive tape on the skin of the subject. The method further comprises identifying nucleic acid sequences in the epidermal sample by using a protein microarray or an enzyme-linked immunosorbent assay (ELISA). The identified nucleic acid sequences are then processed by determining expression levels of a gene associated with irritant contact dermatitis (ICD) or allergic contact dermatitis (ACD). US '165 B2 involves mechanical, thermal, and chemical alteration to DNA extraction protocols to yield higher levels of DNA. These DNA extraction protocols are sufficient to conduct a methylation analysis from the identified nucleic acid sequences.

A co-pending application WO 2023/037103 A1, describes a method of efficient extraction of DNA from skin cells. The skin cells are provided non-invasively using commercially available adhesive tapes. The co-pending patent application further describes biomarkers which are associated with skin ageing and diseases, such as skin cancer (melanoma). The biomarkers are methylated nucleic acid sequences identified by whole genome methylation sequencing. The biomarkers are incorporated into a targeted gene sequencing panel for analyzing specific mutations in regions of interest in the skin cells. The contents of this application are incorporated hereby by reference. The co-pending patent application further discloses the use of the method to extract and process the non-invasive samples for DNA methylation analysis for discovery of biomarkers. Whole genome methylation analysis was used to identify differential biomarkers associated with different types of skin conditions and this specific analysis enabled development of a custom biomarker panel.

Currently a large number of subjects are referred for invasive sampling. In this application we derive data from a study in partnership with Guy's hospital in London. Biopsies were carried out on subjects in Guy's Hospital in London for determining melanoma incidence on the subjects. It was found that 1 in 12 subjects set to the biopsies have melanoma. The rest of the subjects have to undergo further biopsies to find out whether the rest of the subjects have a negative result. The Cancer Research UK found out that 16 700 new melanoma skin cancer cases in the UK are diagnosed every year by invasive sampling (https://www.cancerresearchuk.org/health-professional/cancer-statistics/statistics-by-cancer-type/melanoma-skin-cancer #heading-Zero). Donohue in UW Medicine, “Most mole biopsies are benign, says text analysis of EMRs,” Nov. 9, 2017, states that physicians Drs. Michael Piepkorn and Joann Elmore found out that melanocytic samples accounted for 23 percent of biopsy diagnoses.

International patent application WO 2022/272120 A1 (Horvarth, University of California) discloses that DNA methylation profiles have been used to develop biomarkers for aging. The biomarkers are known as epigenetic clocks and predict chronological age and show promise for inferring health status as an indicator of biological age. WO'120 A1 discloses a method for determining an epigenetic age of a biological sample from a mammal. The biological sample is, for example, blood, or saliva. The method further comprises obtaining genomic DNA from the mammal, observing CpG methylation of the genomic DNAm of a group of at least 40 methylation markers present in genomic polynucleotides and correlating methylation observed in the methylation markers with the epigenetic age of the mammal.

A related paper, Horvath: DNA methylation age of human tissues and cell types. Genome Biology 14:R115, discloses that a base data used comprises 8000 samples with publicly available datasets. Array data is obtained from Illumina 450K and 27K BeadChip platforms. Data processing for training is further disclosed. This training comprises calibration of the chronological age on 21,369 CpG present in the afore-mentioned array data from Illumina 450K and on the 27K BeadChip. The model training used in the method is an Elastic net regularization (GLMNET).

US Patent application No. US 2022/002809 A1 (Horvarth, University of California) discloses a method of obtaining information on mean telomere length in an individual. The method comprises observing methylation of methylation markers in genomic DNA from the individual and correlating methylation observed with mean telomere length, such that information on mean telomere length in the individual is obtained. The method of US 2022/002809 A1 comprises providing a sample from leukocytes. Array data is obtained from Illumina 450K and 850K BeadChip platforms, with a focus on the subset of 450,161 CpG sites. Data processing for training is further disclosed. The data processing comprises normalizing raw DNA methylation data using the noob normalization method.

US Patent application No. US 2020/190568 A1 (OneSkin Tech) relates to systems, software, and methods for gerontological classification of subjects based on a detection of a plurality of epigenetic markers such as methylation status of nucleotides (e.g., CpG) in the genomic DNA. The method of US 2020/190568 A1 comprises providing a sample from skin biopsies, such as from the inner arm, the abdomen, or the ear. US 2020/190568 A1 discloses, in particular, that base data used comprises 508 samples (40 Dermis, 146 Epidermis, 322 Whole Skin Tissue. US 2020/190568 A1 discloses publicly available datasets of GSE51954, E-MTAB-4385, and GSE90124. Array data is obtained from an Illumina 450K BeadChip platform. The method comprises bisulfite conversion. The method also comprises a data preparation comprising using beta values and wherein the samples were quantile normalized. After the normalization, some of the CpG sites are filtered out based on different rules, such as, for example, no sex chromosomes. Data processing for training is further disclosed. The data processing comprises CpG feature selection in three methods through the R software Feature Selection. The CpG feature selection enables removing highly correlated features and keeping only highly significant individual CpG sites (see Boroni, M., Zonari, A., Reis de Oliveira, C. et al. Highly accurate skin-specific methylome analysis algorithm as a platform to screen and validate therapeutics for healthy aging. Clin Epigenet 12, 105 (2020). https://doi.org/10.1186/s13148-020-00899-1).

The three methods tested are GLMNET, XGBoost, ranger. Model training used comprises picking probes by applying three rules: the top 100 probes per method, the top 2000 probes based on the union-importance, and the top 400 probes most correlated with age. The model training further comprises further removing CpG sites, that, albeit highly correlated with age, are further very correlated with each other. The model training used in the method is further an Elastic net regularization (GLMNET).

US Patent application No. US 2021/381051 A1 (Horvarth, University of California) discloses that DNA methylation (DNAm) based biomarkers of aging have been developed for many tissues and organs. However, these biomarkers have sub-optimal accuracy in skin cells, fibroblasts and other cell types that are often used in ex vivo studies. US 2021/381051 A1 analyzed DNA methylation array data sets derived from multiple sources of DNA. US 2021/381051 A1 further discloses a DNAm age estimator based on 391 CpG sites for human fibroblasts, keratinocytes, buccal cells, endothelial cells, lymphoblastoid cells, skin, blood, and saliva samples. The application of this age estimator to ex vivo cell culture systems revealed that cellular population doubling is generally accompanied by an increase in epigenetic aging. US 2021/381051 A1 further discloses a skin & blood clock useful for ex vivo and in vivo studies of human aging. The method of US 2021/381051 A1 comprises providing a sample from human fibroblasts, keratinocytes, buccal cells, endothelial cells, lymphoblastoid cells, skin, blood, and saliva samples. US 2021/381051 A1 discloses base data used comprises thousands of samples with publicly available datasets. Array data is obtained from Illumina 450K and 850K BeadChip platforms. Data processing for training is further disclosed. The data processing comprises normalizing raw DNA methylation data using the noob normalization method. A model training used in the method is an Elastic net regularization (GLMNET).

There is therefore a desire to determine a stage of an epigenetic property of an epidermis, preferably non-invasively.

There is a benefit for improving sample acquisition to a method that samples a homogenous but functionally relevant cell type, is non-invasive, pain free and can be performed without a trained clinician.

### SUMMARY OF THE INVENTION

A method and system for determining a stage of an epigenetic property of an epidermis of a subject is taught in this disclosure.

The method comprises providing a keratinocyte sample from the subject, followed by identifying methylated nucleic acid sequences in the keratinocyte sample by enzymatic methyl sequencing (EM-seq). In one aspect, the keratinocyte sample is provided non-invasively from the subject. The identification of the methylated nucleic acid sequences enables a targeted epigenetic analysis of the keratinocyte sample. The method further comprises using a trained machine-learning model to process the identified methylated nucleic acid sequences and thereby to determine the stage of the epigenetic property.

The provision of the keratinocyte sample involves taking the sample from an outermost layer of the skin of the subject which is composed substantially of 90 to 95% keratinocytes. The method does not involve taking full thickness biopsies.

The methylated nucleic acid sequences used as training samples originate directly from subjects. The obtention of the training data from subjects is facilitated by a willingness of the subjects to voluntarily provide the keratinocyte samples.

The method, as set out in this disclosure, enables identification of melanoma and other diseases of the epidermis at an early stage. This means that a smaller number of patients will require an invasive biopsy. The method therefore contributes to reducing an overall burden on medical service, as the method enables a reduction of unnecessary invasive biopsies, such as those used in the past for clinical trials for product development. This method also eliminates the need for bisulfite conversion of methylated DNA which has been used to enable the distinction of methylated cytosines from unmethylated cytosines. In one aspect, the methylated nucleic acid sequences are methylated deoxyribonucleic acid (DNA).

A computer-implemented method for training the machine-learning model is taught in this disclosure. The computer-implemented method comprises receiving a training dataset comprising a plurality of data elements. The data elements comprise methylated nucleic acid sequences, identified by enzymatic methyl sequencing (EM-seq), associated epidermal properties, and epidermal stage results.

The trained machine-learning model applies a principal component analysis to the plurality of data elements. In one aspect, an elastic net regression model is used for training the machine-learning model.

The system comprises an input device for inputting at least one methylated nucleic acid sequence identified by enzymatic methyl sequencing. The system further comprises a memory for storing a trained machine learning model and a processor for using the inputted at least one methylated nucleic sequence and the stored trained machine learning model to determine the stage of the epigenetic property.

The epigenetic property is, for example, melanoma or skin ageing. The system and method of this document can be used, for example, in determining the damage from ultraviolet light to skin. The system and the method can further be used in tracking a response to therapeutic interventions.

The system and the method can be used in determining if a melanoma drug worked on the subject. The system and the method can be used in determining if an oral drug that targets a liver disease with psoriasis symptoms worked on the subject. The system and the method can further be used in determining if a skincare topical product worked on the subject.

### DESCRIPTION OF THE FIGURES

FIG. 1 shows a view of a system for determining a stage of an epigenetic property of a subject.

FIG. 2 shows a flow chart describing a method for determining the stage of the epigenetic property of the subject.

FIG. 3 and FIG. 4 show a flow chart describing a computer-implemented method for training a machine-learning model for determining the stage of the epigenetic property.

FIG. 5 shows a PCA plot of CpG sites using a binomial elastic net model for the training dataset indicating different stages of melanoma.

FIG. 6 shows a heatmap of probabilities for nevi, for primary melanoma and for metastatic melanoma.

FIG. 7 shows a plot of distribution of the log odds of the stages of melanoma over different stages of melanoma for the training dataset.

FIG. 8 shows a PCA plot of CpG sites selected by an elastic net multinomial model for the training dataset comprising different stages of melanoma.

FIG. 9 shows a heatmap of probabilities for nevi, for primary melanoma and for metastatic melanoma.

FIGS. 10A and 10B show graphs of log odds of CpG sites selected by the elastic net multinomial model for the training dataset and for a validation dataset, respectively. FIG. 10A and FIG. 10B are plotted over different stages of melanoma.

FIG. 11 shows a histogram of age distribution for a training dataset comprising different biological ages.

FIGS. 12A and 12B show PCA plots of CpG sites for different chronological ages of the subjects. FIG. 12C shows the predicted age over an actual age of the subjects. 288 CpG sites where methylation is correlated specifically with ageing in skin, i.e., CpG model, were identified in the training model and are plotted on FIGS. 12A, 12B and 12C.

FIGS. 13A and 13B show PCA plots of correlated CpG sites for different chronological ages of the subjects. FIG. 13C shows the predicted age over the actual age of the subjects. FIGS. 13A, 13B and 13C show a principal component model.

FIG. 14 shows a graph of the principal component model over the CpG model.

FIG. 15 is an example of the training dataset.

FIG. 16 shows an example of a PCA analysis of the dataset.

FIG. 17 shows the results of the tSNE algorithm using all M Values.

FIG. 18 shows a size distribution of regions retrieved by Twist's probes.

FIG. 19 shows a density distribution of the log 10 distance between a region and a next downstream region per chromosome.

FIG. 20 (left) shows a density distribution of the log 2 count of the CpG sites per region defined in a region probe. FIG. 20 (right) shows a density distribution of the number of the CpG sites per region when accounting for the length of the region itself.

FIG. 21 shows a train/test dataset distribution of transformed ages. The training dataset is shown on the left, the test dataset is shown on the right.

FIG. 22 shows prediction values in the training dataset. The top plot is the predicted value vs real value per sample, and the bottom plot is the distribution of residuals.

FIG. 23 are prediction values in the testing dataset. The top plot is the predicted value vs real value per sample, and the bottom plot is the distribution of residuals.

FIG. 24 shows prediction using the present method on new samples from the targeted sequencing project (n=73).

FIG. 25 shows independent biological replicates indicating small residual error.

### DETAILED DESCRIPTION OF THE INVENTION

The invention will now be described on the basis of the drawings. It will be understood that the embodiments and aspects of the invention described herein are only examples and do not limit the protective scope of the claims in any way. The invention is defined by the claims and their equivalents. It will be understood that features of one aspect or embodiment of the invention can be combined with the feature of a different aspect or aspects and/or embodiments of the invention.

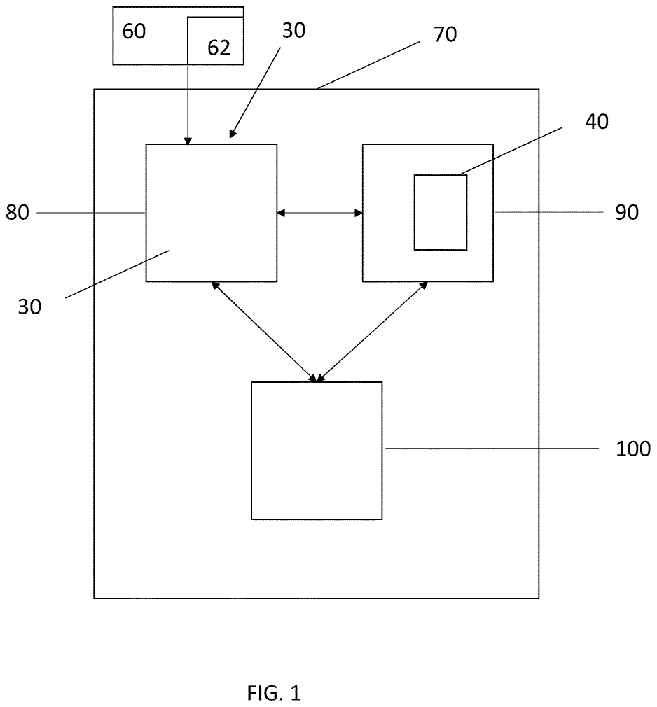

FIG. 1 shows a view of a system 70 for determining a stage of an epigenetic property of a subject 10, such as a patient. The system 70 comprises an input device 80, a memory 90 and a processor 100 which are connected to each other. The memory 90 includes a trained machine-learning model 40. The creation of the machine-leaning model 40 will be described later.

The input device 80 is used to input data into the system 70. As will be described later, the data includes data elements relating to methylated nucleic acid sequences 30 of a keratinocyte sample 20 which is extracted from the subject 10 as well as one or more training datasets 60.

The methylated nucleic acid sequences 30 are identified by enzymatic methyl sequencing (EM-seq). EM-seq was studied by Vaisvila R et al. in “Enzymatic methyl sequencing detects DNA methylation at single-base resolution from picograms of DNA”, Genome Res. 2021 July; 31 (7): 1280-1289. Enzymatic methyl sequencing (EM-seq) is a technique that uses deoxyribonucleic acid (DNA) methyltransferases to detect DNA methylation in the keratinocyte sample 20. The technique involves labelling the DNA from the keratinocyte sample 20 with a methyltransferase enzyme that adds a methyl group to the DNA in the keratinocyte sample 20 at specific sites within the DNA to produce so-called “labelled DNA”. The labelled DNA is then sequenced to determine the location of the methylated sites within the DNA.

The methylated nucleic acid sequences 30 can, in addition to the methylated (labelled) DNA, comprise methylated ribonucleic acid (RNA), or a combination thereof. The methylated DNA comprises, in one example, a methylated cytosine consecutive to a guanine (termed a CpG site). The methylated cytosine can be, for example, 5-methylcytosine (5mC), 5-hydroxymethylcytosine (5hmC), 5-formylcytosine (5fC) or 5-arboxylcytosine (5caC).

The methylated DNA comprises, in another example, a methylated adenine. The methylated adenine can be, for example, N6-methyladenine (6 mA).

The processor 100 executes the machine learning model 40 and uses the methylated nucleic sequences 30 with the stored trained machine learning model 40 to determine the stage of an epigenetic property. The epigenetic property can be, for example, melanoma or skin ageing.

The stage of the melanoma can be, for example, nevi (i.e., no melanoma), primary melanoma or metastatic melanoma, or can be divided into substages as will be explained later.

The stage of the skin ageing can be, for example, a biological age of the subject 10. The concept of “biological age” refers to an extrinsic epidermal age of the subject 10, i.e., ageing that is influenced by an environmental exposure of the subject 10. The biological age is independent of the chronological age, i.e., true age of the subject. The biological age is a measure of how the environment is changing normal ageing processes of the skin of the subject. The inventors have found that biological ageing, for instance, is higher in subjects with melanoma. Berstein et al. in “Assessing the causal role of epigenetic clocks in the development of multiple cancers: a Mendelian randomization study” eLife 2022, https://doi.org/10.7554/eLife.75374 found out that the identification of the biological ageing can also be used to predict the phenotype of the skin sample.

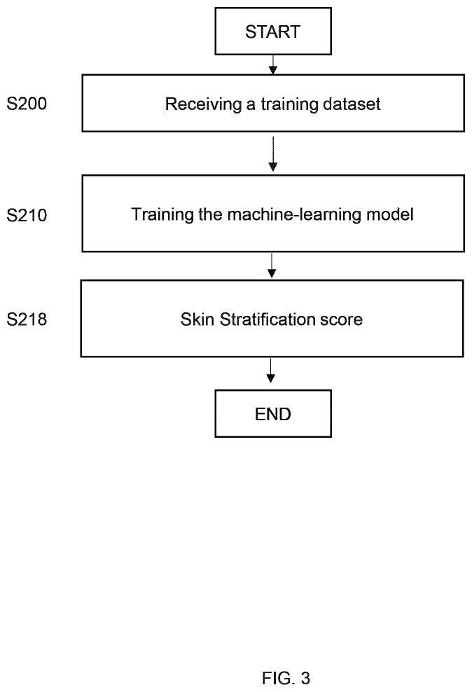

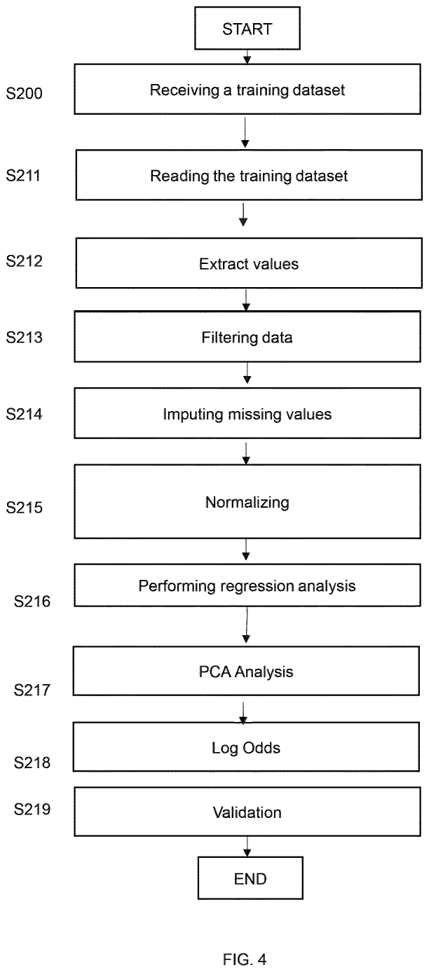

The training of the machine learning model 40 will now be described. FIG. 3 shows a flow chart describing the training of the machine-learning model 40 and FIG. 4 sets out the steps in more detail.

The method comprises a step S200 of receiving a training dataset 60 through the input device 80. The dataset 60 comprises a plurality of data elements 62. The dataset 60 is generated both from publicly available datasets and datasets which are generated internally.

In one aspect, the data elements 62 comprise methylated nucleic acid sequences 30 originating, for example, from the Gene Expression Omnibus (GEO) database. The data elements 60 can be a Genomic Spatial Event (GSE) database. This database is maintained by the US National Center for Biotechnology Information (NCBI) and collates biological information submitted by researchers. In particular, the GSE database comprises gene sequences of keratinocyte samples collected from biopsies or collected from skin patches.

In one non-limiting example, the training data sets used were GSE120878 and GSE86355. The training data set GSE120878 was derived from formalin fixed paraffin embedded (FFPE) biopsies and had 89 samples with melanoma and 73 samples indicating nevi. The samples were collected at the University of North Carolina or the University of Rochester between 2001 and 2012.

The training data set GSE86355 was derived from fresh-frozen biopsies and had 61 samples of melanoma, of which 33 were primary samples and 28 metastatic samples, together with 14 samples indicating nevi. The samples were collected at KU Leuven.

In a further aspect, the data elements 62 comprise methylated nucleic acid sequences 30 which originate from self-generated training samples 20.

It will be appreciated that the aim of this machine-learning model is to create a model which is independent of the methods of fixation and other lab-based processing techniques.

The data elements 62 further comprise epidermal properties and epidermal stage results associated with the methylated nucleic acid sequences 30. The epidermal stage results are identified by a clinician by using histopathology data from the samples.

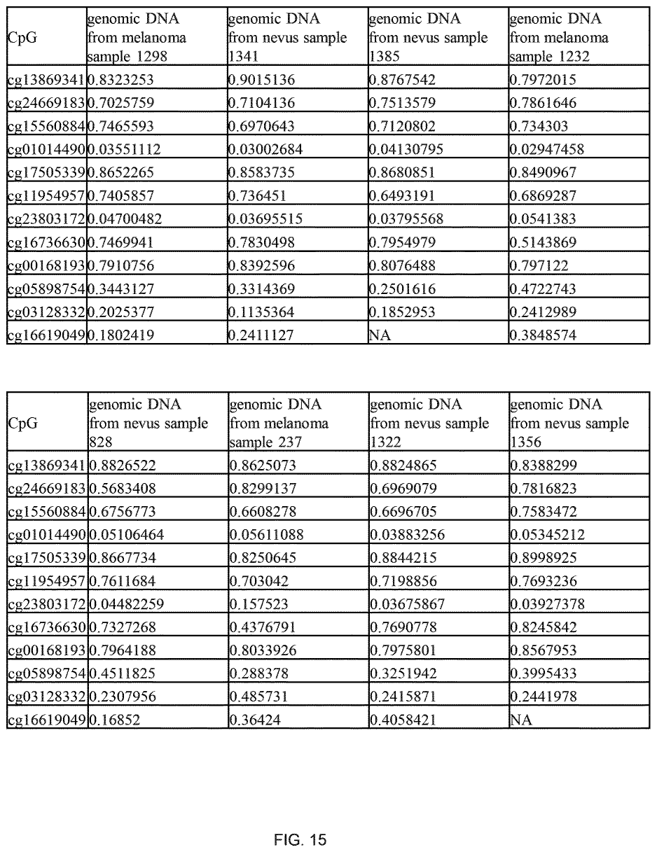

Examples of the data elements 62 from the dataset are shown in FIG. 15. The rows in the data elements 62 comprise methylated CpG sites, i.e., biomarkers, associated to melanoma. The methylated CpG sites originate from GEO datasets, from Cancer Genome Atlas (TGCA) datasets and from trained datasets. The columns show their occurrences in percentages in the DNA of the datasets.

In the training step S210, the machine-learning model 40 was trained using the data elements 62 from the inputted training dataset 60, as described in “Minfi: a flexible and comprehensive Bioconductor package for the analysis of Infinium DNA methylation microarrays” by Aryee et al., 2014, Bioinformatics, 30 (10), 1363-1369, doi:10.1093/bioinformatics/btu049.

Two types of machine-learning model 40 were used. The machine-learning model 40 is stored on a GitHub repository. The binomial machine-learning model 40 was computed solely from the keratinocyte samples provided non-invasively, compared to previously developed machine-learning model which involved both epidermal markers and dermal markers derived from biopsy. The binomial machine-learning model 40 aims to classify differences between melanoma samples and nevi samples. This binomial machine-learning model 40 is naïve to the melanoma samples being primary melanomas or metastatic melanomas. The binomial machine-learning model 40 simply looks for methylation differences between the melanoma samples and the nevi samples.

A multinomial machine-learning model 40 was used to distinguish between the melanoma samples being primary melanoma samples or metastatic melanoma samples. The multinomial machine-learning model can leverage, for example, the additional biopsy date found in the GSE86355 dataset.

In one example, the machine-learning model 40 is trained using an elastic net binomial model. The model building was performed using the glmnet package (stored at Stanford University) for the R statistical programming software. The use of this package is, however, not limiting of the invention and other packages can be used. The elastic net multinomial model originates from Friedman et al., Regularization Paths for Generalized Linear Models via Coordinate Descent. Journal of Statistical Software, 2010, 33(1), 1-22, https://www.jstatsoft.org/v33/101/.

The training step 210 is carried out as follows. The data elements 62 of the dataset 60 are read into the processor 100 in a reading step S211. The beta values 62b and the corresponding values of the epidermal stage 62s are extracted in an extraction step S212 from the training dataset 60. The beta values 62b are commonly used to indicate the degree of methylation in a sample of DNA. The beta value 62b represents the ratio of the methylated signal intensity to the total signal intensity at a specific locus in the DNA sequence of the sample (in this case the keratinocyte sample 20). The beta value 62b ranges from 0 (i.e., the DNA sequence is completely unmethylated) to 1 (i.e., the very rare case in which the DNA sequence would be completely methylated). In other words, the beta value enables quantification of the proportion of methylated CpG sites in the DNA in the keratinocyte sample 20. A beta value of 0.57 would, for example, mean that 57% of the sites are methylated.

The training step S210 further comprises a filtering step S213 of filtering the data elements 62 in the training dataset 60 to removing the data elements from the training dataset 60 with CpG probes that have a single nucleotide polymorphism (SNP) which is +/−two base pairs from the CpG and removes these data elements 62. The filtering step S212 removes further those data elements 62 that represent DNA sequences in the dataset 60 that cross hybridize to multiple genomic locations and/or are located on the X and Y chromosomes.

The reasons for the removal of SNPs are as follows. If a probe contains an SNP on or very close to its CpG then the probe can cause different methylation values depending on whether or not that SNP is present. This can happen, for example, if the CpG site in the normal population has the base pairs CG and is 100% methylated. If the C mutates to a T, then the C (T) can no longer be methylated and therefore would give a value of 0% or 50% (50% if the SNP were heterozygous—i.e., only occurred on one allele.) It is therefore necessary to remove this data element 62.

The cross-hybridizing probes are probes that are on the arrays that were found to map to multiple genomic locations, i.e., a probe map target its real CpG on chromosome 1 but may also unintentionally target another CpG on chromosome 2. This makes the readings from these probes unreliable and as such it is advised that they are removed from the analysis. This is explained in Chen et al, “Discovery of cross-reactive probes and polymorphic CpGs in the Illumina Infinium Human Methylation450 microarray”, Epigenetics, Vol 8, 2013, pages 203-209, doi:10.4161/epi.23470

The probes on X and Y chromosomes are removed because females lack a Y chromosome so if the final model were to use any Y chromosome probes it would not be useful for classification of melanoma in females.

The removal of SNPs, the removal of the cross hybridization and the removal of X and Y chromosomes are described by Peters et al., “De novo identification of differentially methylated regions in the human genome”, Epigenetics & Chromatin, 2015, 8:6, doi:10.1186/1756-8935-8-6.

Data resulting from the filtering step S213 can lack uniformity in some regions of fragments of samples. Gaps can occur between the fragments of the samples. The structure of the fragments needs to be estimated so that an indication of the location of the gaps between the fragments of DNA can be predicted. The filtering step S213 is therefore followed by a missing value calculation step S214 which uses a k-nearest neighbors (KNN) imputation method. The KNN imputation method aims at identifying ‘k’ samples in the dataset that are similar or close in the space. The identification of the ‘k” samples enables to estimate the location of possible gaps on the fragments, i.e., missing data points of the data elements 62. The missing data points are imputed missing values imputed using the mean value of ‘k’-neighbors found in the training dataset 60.

The training S210 further comprises a normalizing step S215 in which the training dataset 60 is normalized. The normalization step is used because the arrays have two types of chemistry and it is advisable to normalize them to make the data from the arrays to be statistically comparable, as explained by Mueller et al. in “RnBeads 2.0: comprehensive analysis of DNA methylation data”, Genome Biology, 2019, 20:55, available at https://genomebiology.biomedcentral.com/articles/10.1186/s13059-019-1664-9. The training S210 is also explained by Teschendorff et al., “A beta-mixture quantile normalization method for correcting probe design bias in Illumina Infinium 450 k DNA methylation data”, Vol. 29 no. 2 2013, pages 189-196, doi:10.1093/bioinformatics/bts680.

Beta Mixture Quantile Normalization (BMIQ) is used to normalize the distribution of the data from the different chemistries.

The normalization step S215 is followed by an analysis step S216 which involves performing a cross-validated regularized logistic regression analysis on the training dataset 60. The analysis step S216 comprises removing the data elements 62 with CpG sites with low coverage from the training dataset 60. Low coverage would imply that the machine-learning model 40 needs to be fine-tuned to predict the methylation on the CpG sequence of the training dataset 60. High coverage is essential for a good estimation of the methylation on the CpG sites. The analysis step S216 further comprises calculating quantiles of the resulting CpG sites of the training dataset 60 and filtering outliers CpG sites that do not fit the distribution of the resulting CpG sites. The analysis step S216 further comprises performing a logistic regression with cross-validation on the filtered CpG sites. The cross-validated regularized logistic regression analysis in the analysis step S216 is carried out to remove the risk of overfitting of the machine-learning model 40.

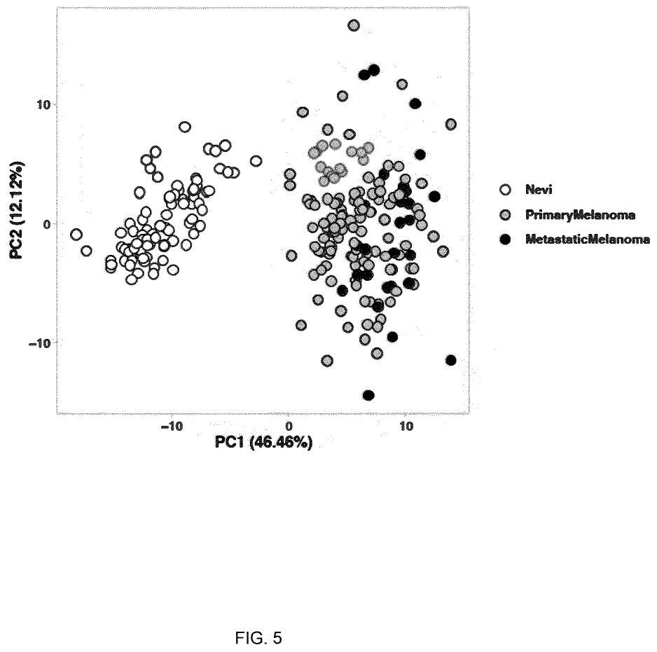

Finally, those data elements 62 representing the methylated CpG sites selected after the analysis step S216 are subjected to a principal component analysis in a PCA step S217 to identify underlying patterns and correlations among the data elements 62 of the data set. The result of the PCA step S217 is shown on the PCA plot of FIG. 5 using the binomial elastic net machine learning model 40. FIG. 5 shows that the second principal component PCA2 on the y-axis captures the differences between the nevi and the melanoma. There is also some variance between the primary melanoma and the metastatic melanoma.

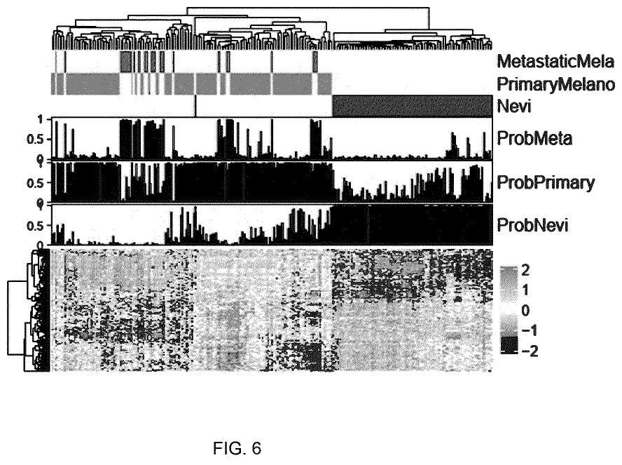

The training step S210 further comprises converting in a probability conversion step S218 the log odds (logit) of the training dataset 60 into probabilities using an exponential function. FIG. 6 illustrates the probabilities determined after the probability conversion step S217 in a heatmap with different greyscales for nevi, for primary melanoma and for metastatic melanoma.

General data visualization is described by Wickham et al in “ggplot2: Elegant Graphics for Data Analysis. Springer-Verlag New York, 2016” and by Gu et al., (2016) “Complex heatmaps reveal patterns and correlations in multidimensional genomic data”, Bioinformatics, https://doi.org/10.1093/bioinformatics/btw313.

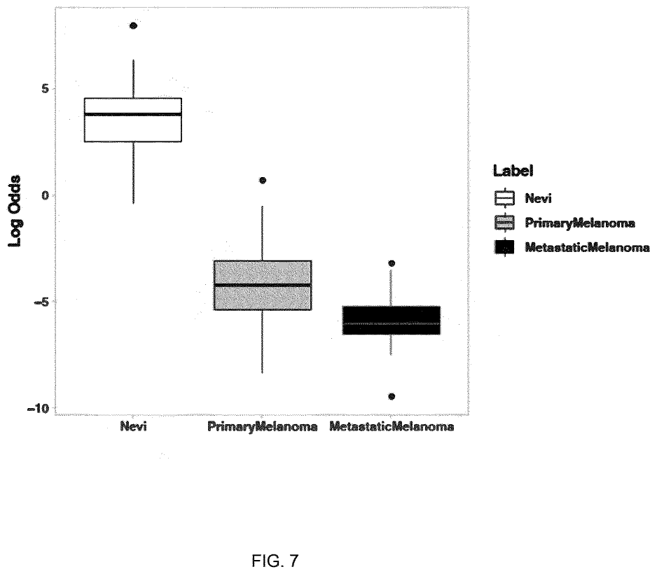

FIG. 7 shows a plot of distribution of the log odds of the stages of melanoma, i.e., nevi, primary melanoma, and metastatic melanoma over the different stages of melanoma.

In a second example, the machine-learning model 40 is trained in the step S210/S216 using an elastic net multinomial model, i.e., an elastic net regression model.

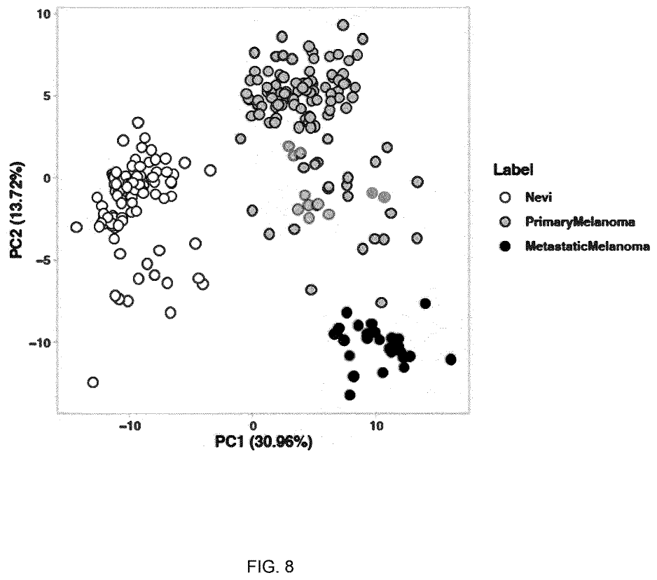

FIG. 8 shows a PCA plot of CpG sites selected by the elastic net multinomial model in the second example for the training dataset 60 which includes different stages of melanoma. It can be seen from FIG. 8 that the elastic net multinomial model clearly distinguishes between the nevi, the samples with the primary melanoma and the samples with the metastatic melanoma.

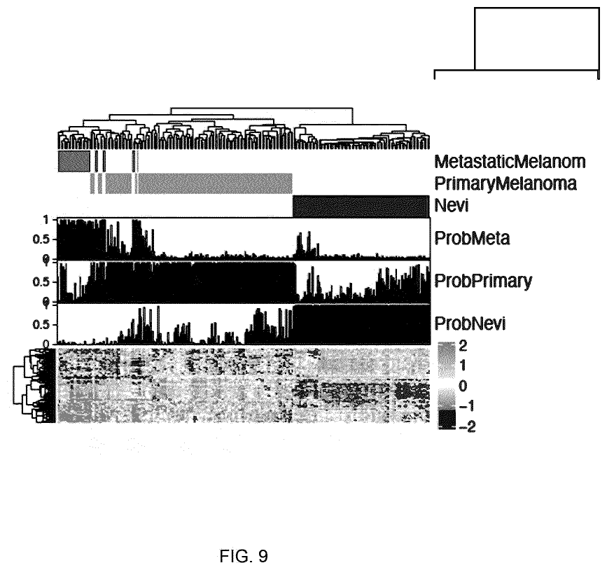

FIG. 9 illustrates the probabilities determined after step S216 of the elastic net multinomial model of the second example in a heatmap with different greyscale for nevi, for primary melanoma and for metastatic melanoma.

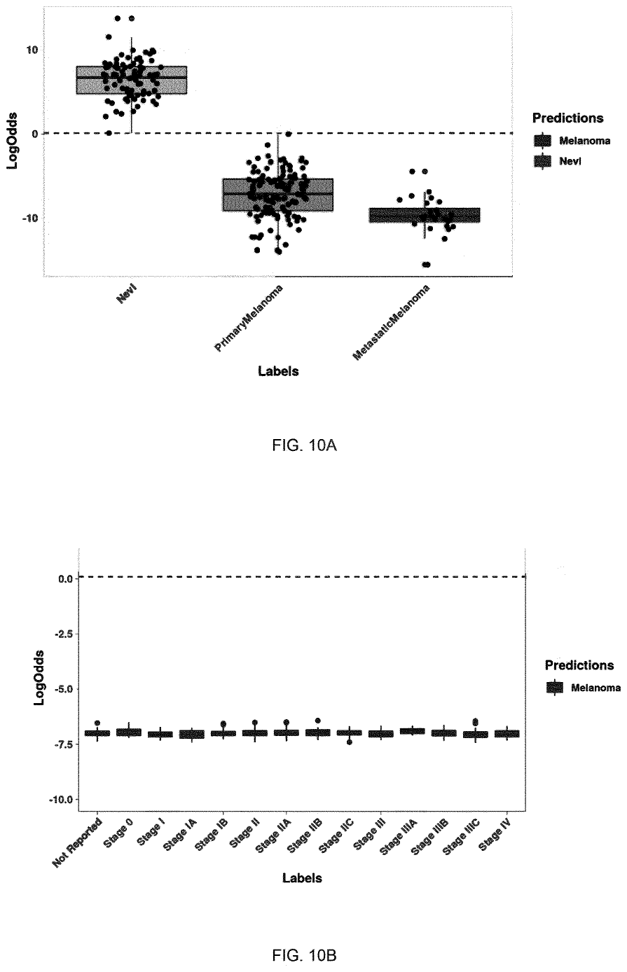

FIG. 10A shows the predicted log odds of the CpG sites selected by the elastic net multinomial model for the training dataset 60. FIG. 10A is plotted over nevi, primary melanoma, and metastatic melanoma.

The training step S210 can be completed by a validation step S219 to evaluate the validity of the machine-learning model 40 trained in the steps S211 to S216. The validation S219 comprises receiving in a validation receiving step S120 a validation dataset 110. The validation dataset 110 is similar or identical in construction to the training dataset 60 and comprises similar elements.

The validation set 110 used in this example was TCGA-SKCM from the Cancer Genome Atlas established by the US-based National Cancer Institute. This data set had 473 samples of melanoma, of which 105 indicated primary cancers and 367 indicated metastatic cancers. However, the validation dataset 110 included no samples with nevi.

In other cases, the validation dataset 110 can be a subset of a dataset which was used for the training step S210. For example, 80% of the data elements 62 in the dataset 60 are used for training the machine-learning model 40 and the remaining 20% of the data elements 62 are used for validating the machine learning model 40.

FIG. 10B shows the determined stages of melanoma from the validation set 110. FIG. 10B is a deconvolution of FIG. 10A. FIG. 10B shows three different stages of melanoma.

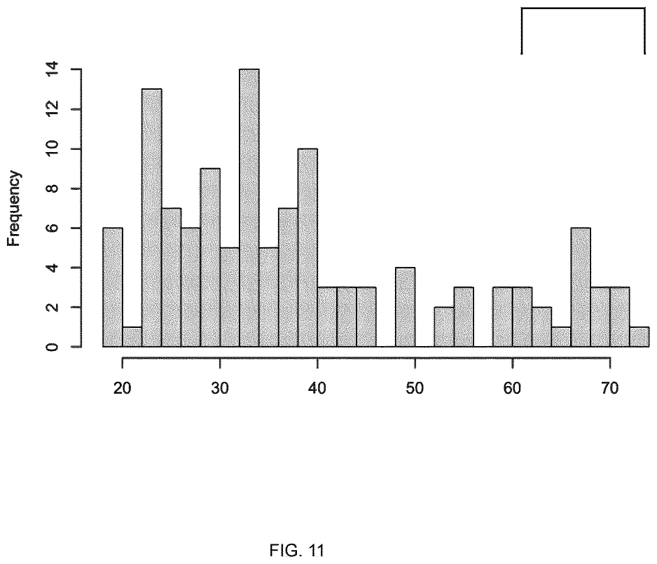

In FIG. 11B, each of the three different stages can be further divided into up to four subtypes, for example designated by letters A through D. The higher the stage and the letter, the more severe the prognosis of melanoma. For example, stage IIC is considered more serious than stage IIB.

At stage 0, the melanoma is malignant. The malignant melanoma is confined to a top layer of the keratinocyte sample 20 and only exists in the epidermis. There is no indication that a tumor of melanoma has spread to lymph nodes of the keratinocyte sample 20. At stage I, the tumor is localized and has begun to spread from the epidermis to the dermis of the keratinocyte sample 20. There is no evidence of metastasis of the tumor. The stage I is divided into stage IA and stage IB. The melanomas at the stage I are typically less than 2 mm thick. At stage II, the tumor has spread to both the dermis and epidermis of the keratinocyte sample 20. The stage II is further divided into subtypes IIA, IIB, and IIC.

At stage III, the tumor has spread to one or more regional lymph nodes or has developed deposits of melanoma in the skin or in the dermis of the keratinocyte sample 20 along the lymphatics prior to reaching a lymph node. This stage is also known as an in-transit or satellite metastasis. There is no indication that the tumor has spread to other sites at this stage. This stage is divided into four subtypes III A, III B, III C and III D. At stage IV, the tumor, i.e., cancer, has spread beyond the top layer and the regional lymph nodes to other areas of the body of the subject 10. In the stage IV, the most common sites of metastasis are distant skin and lymph nodes, then lungs, liver, brain, bone, and/or intestines.

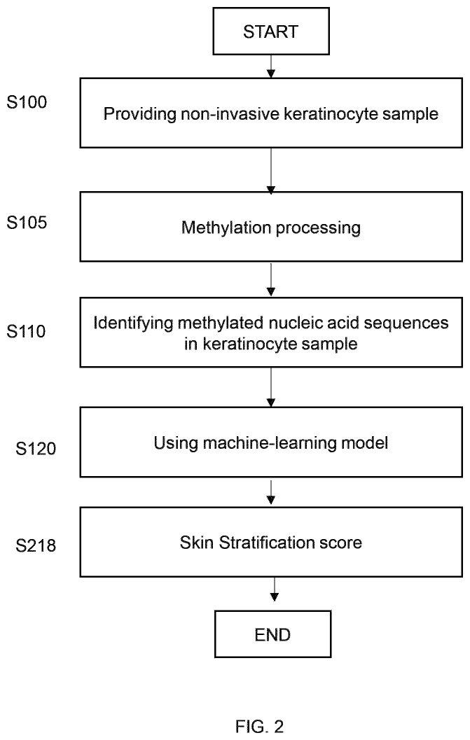

Once the machine-learning model 40 has been programmed, the system 100 can be used to determine the stage of the epigenetic property of the subject. This is set out in FIG. 2. The method comprises an extraction step S100 in which a keratinocyte sample 20 is extracted from the subject 10 and the DNA from the keratinocyte sample 20 is extracted. The extraction step S100 is carried out in one aspect non-invasively and can be carried out using, for example, an adhesive tape.

The method further comprises a methylation step S105 in which the DNA in the keratinocyte sample is methylated. An identifying step S110 identifies the methylated nucleic acid sequences 30 in the keratinocyte sample 20 and the identification step S110 is followed by using in processing step S120 the trained machine-learning model 40 to process the identified nucleic acid sequences 30. In one non-limiting example, this is done by Illumina NovaSeq S4 flow cells and thereby determine in a determination step S130 the stage of the epigenetic property.

In one example, the method set out in this document is used for determining a skin age of the subject 10. The skin age is also known as an epigenetic clock.

FIG. 11 shows a histogram of age distribution of a training dataset 60 created using a software module from the R project for statistical computing.

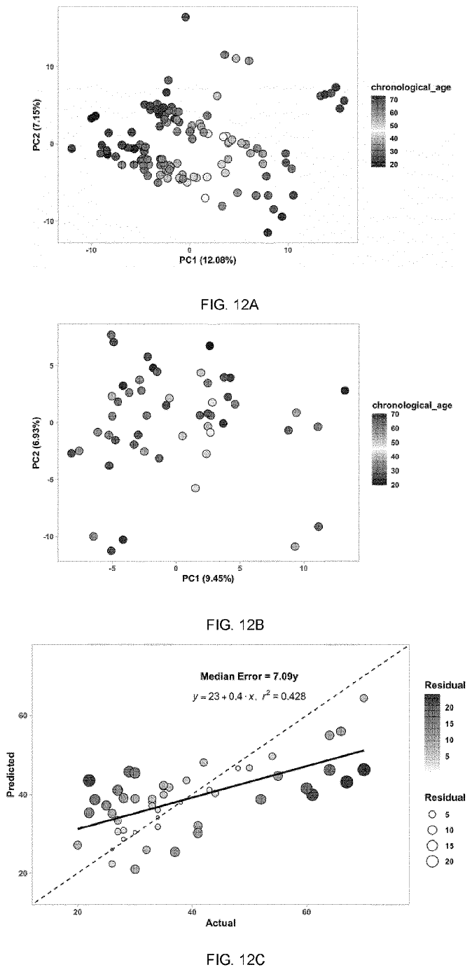

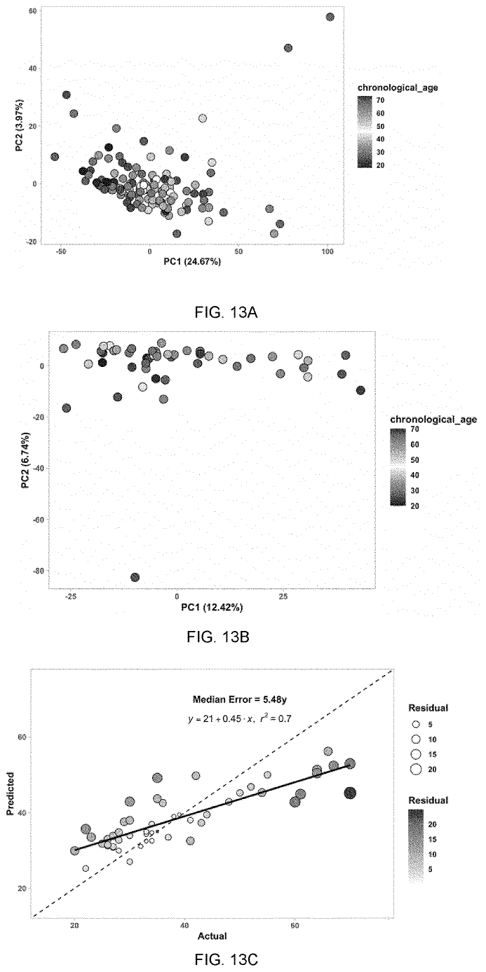

FIGS. 12A and 12B show PCA plots for 288 CpG sites where methylation is correlated with the skin ageing. It has been found by the inventors that there are 288 CpG sites at which the methylation is correlated with the ageing of the skin. FIGS. 12A and 12B are used to build the trained machine-learning model 40. The skin ageing in FIG. 12C shows a plot of the predicted skin age, i.e., chronological age over the actual skin age of the subjects. FIGS. 13A and 13B show PCA plots for principal components. The principal components refer to the variables in the training dataset 60 that differentiates the dataset the most.

FIGS. 12A and 12B are induced with technical variance due to batch effects. Batch effects mostly occur when the data elements originate from different EPIC arrays. The problem of the technical variance will be mitigated in the future as sequencing data from custom panels will have been used. FIGS. 13A and 13B show a principal component model. 2500 methylated CpG sites were identified with the principal component model.

FIGS. 12C and 13C show the predicted age on the y-axis against the actual biological age on the x-axis using the various models over the actual age of the subjects. As noted above, the PCA machine-learning model 40 of this document has been found to enrich the dataset and account for any technical variation arising from using multiple sequencing platforms. FIG. 12C shows an error of 7.09 years, whereas FIG. 13C shows an error of 5.48 years.

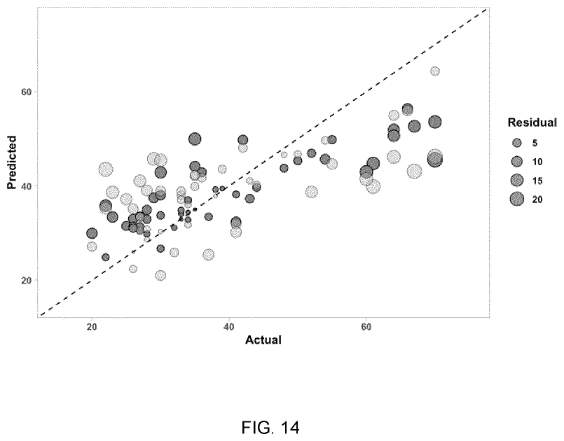

FIG. 14 shows a graph of the chronological age of the subject (human) (as determined from the CpG sites in the sample) on the x-axis and the predicted age on the y-axis using the PCA method set out in this document. The regression line is the dotted diagonal running through the graph.

A further example of the method will now be described. In this aspect, the method comprises providing a keratinocyte sample from the skin epidermis by taking the sample from the face and/or from the forehead from the subject, for example from the uppermost layers of the skin.

Base data used in this example comprises initially 313 keratinocyte samples. The base data was filtered so that 277 samples were obtained. The base data comprises a mix of Whole Genome (WG) data and TWIST Panel data. These data are submitted to enzymatic conversion. This data further forms sequencing data. The WG data are limited to 4M CpG sites in the TWIST Panel data (for more details see “Twist Human Methylome Panel”, https://www.twistbioscience.com/products/ngs/fixed-panels/human-methylome-panel (accessed: 28 Feb. 2024).

Preparation of the data comprises removing chromosome X (chrX), chromosome Y (chrY) and Homo sapiens mitochondrion sequence (chrM) from the samples. The data preparation further comprises removing those values per sample of all CpGs that are covered by less than 7 reads. This removal is followed by removing those CpGs with a high number of missing values. The missing values can occur if there are reads that did not cover a region—In this case, there will be missing values, and this will translate in missing values in the sequencing data. These missing values are not useful in the data analysis and the missing values are therefore removed in the filtering steps The removal of the CpGs with high number of missing values results in removing values in 70% of the sample. The samples with high numbers of missing probes are further removed and result in removing samples with more than 10% of missing values. Removing samples with Avg Twist Coverage under 6 is further conducted. The remaining CpG sites per sample are taken for each region covered by the probes of the Twist panel. Averaging a signal of the level of the DNA methylation observed across the available CpG sites is further conducted.

In a next step, data processing for training is conducted. The data processing comprises dividing data into training sets in train (66.6%) and testing sets (33.3%). The Spearman correlation between the regions and the Transformed Age Values is conducted after the division of the data into the two sets. Those regions that have an absolute Spearman correlation over 0.30 are selected for further use. This calculation of the Spearman calculation enables to enforce even distribution of ages in train data and in test data.

A model training used in the method comprises selection steps with cross-validation in the training data. These selection steps are imputing missing values with average, followed by removing the 10% least variable regions. The selection is devoid from a scaling of data. The model training comprises selecting those highly correlated regions as defined by the Mutual Information Measurement, so that 90% of the most informative regions are kept. Principle Component Analysis (PCA) is performed on these selected (i.e., after filtering) CpG sites. The step of performing the PCA is followed by filtering out the PCAs using the Lasso method. The filtering out is followed by inputting the PCAs into an ElasticNet model with further Cross Validation.

This order of steps results in a total number of CpG sites listed at 14838. The mean absolute error in the test data is 6.53, the median absolute deviation in the test data is 4.12, root-mean-square error (RMSE) in the test data is 9.09 and the R2 score in the test data is 0.69.

More details of the above-mentioned example are now provided.

The Whole Genome (WG) data is a first subset of the sample. The first subset has been through Whole-Genome Sequencing (em-seq) and bioinformatically subsetted to include only the 4M CpG sites in a TWIST Biosciences panel.

The TWIST Panel data is a second subset of the sample. The second subset has been processed using hybridization hybridisation of 4M CpG panel provided by Twist Biosciences. This processing enables facilitating targeted 4M CpG sequencing on Illumina Novaseq S4 flow cells (for the melanoma samples extracted from the forehead).

The two datasets use different laboratory preparation protocols. In a first step, consistency between the two datasets is explored to see whether the datasets allow combination. Two dimensionality reduction methods were used to simplify the highly multidimensional data (approximately 4M CpGs) into two dimensions.

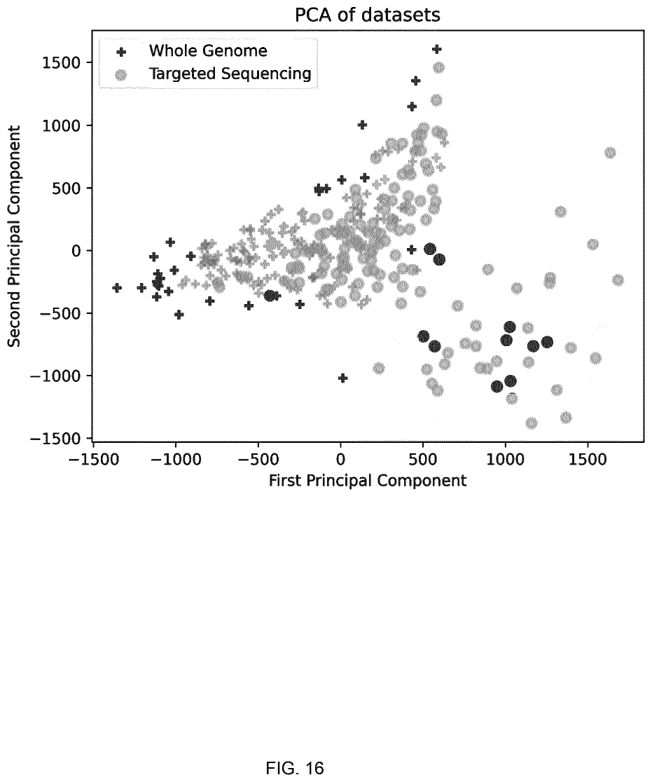

FIG. 16 illustrates a first approach. A Principal Component Analysis (PCA) of the data is performed. It will be known that the method of performing PCA is widely used in the Biological Sciences to reduce the dimensionality and/or explore highly multidimensional datasets, like those produced in RNA-Seq experiments, as well as multiple other types of data (see Greenacre, M., Groenen, P. J. F., Hastie, T. et al. Principal component analysis. Nat Rev Methods Primers 2, 100 (2022). https://doi.org/10.1038/s43586-022-00184-w).

Intuitively, the PCA consists of creating new features as linear combinations of the features already available in the dataset, effectively condensing the information enclosed in multiple features into single columns, called Principal Components (PCs). The first approach also enables identifying how much of the information of the dataset is explained by each of the produced PCs, with the first PC being the most informative. In sequencing the datasets, strong separation of the samples with one of the first PCs is usually associated with notorious technical effects that may obscure the biological information if processing both of the datasets together.

FIG. 16 shows the PCA analysis of the dataset using M Values after a removal of the CpGs sites with no variance, followed by mean imputation in the CpG sites with missing values.

The PCA plot in FIG. 16 shows the distribution of the skin sample data points along the first two principal components that capture the most significant variance in the dataset. The clustering of the data points suggests that although some of the skin samples have a more different distribution in one of the subsets, there still is significant similarities between most of the skin samples between the two groups: those prepared for targeted 4M CpG sequencing and those processed through Whole-Genome Sequencing. This overlap implies that there are no strong technical batch effects separating the datasets, highlighting on one side the consistency of this first approach as well as enabling working with both of the datasets without having to rely on biassing normalization methods. The tight grouping within each of the subsets suggests good internal consistency, while the slight separation between groups could indicate differential methylation profiles or the effect of background noise.

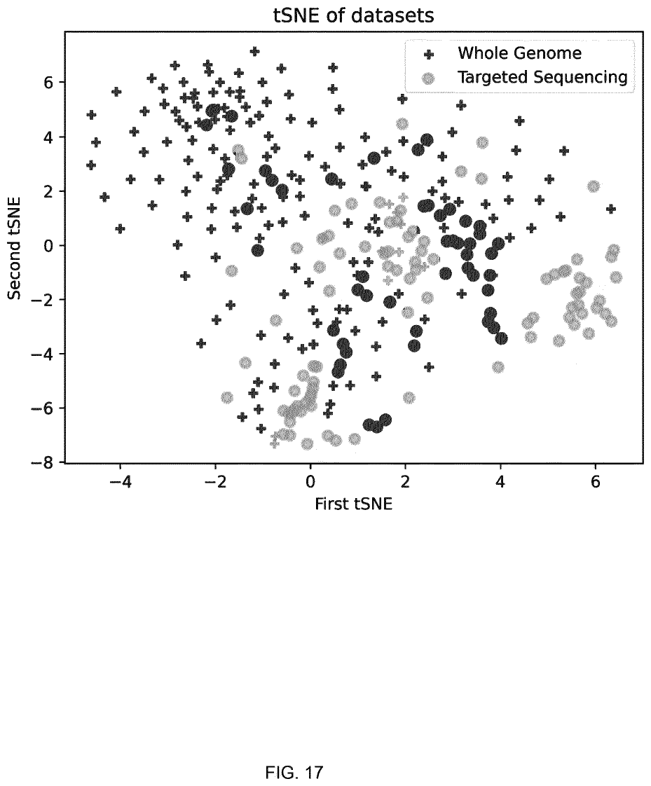

FIG. 17 illustrates a second approach. The second approach comprises exploring the datasets in the t-Distributed Stochastic Neighbor Embedding (tSNE) algorithm. This second approach is not used for dimensionality reduction, but for clustering of the data. The tSNE algorithm does not seek to conserve information, unlike the PCA. The tSNE algorithm seeks to reduce multidimensional datasets into only two or three features that maintain the pairwise similarity between the skin samples and can be used for visualization. The algorithm follows an iterative approach. The tSNE algorithm seeks to cluster together those data points that are more similar and separate those data points that are less similar. Another advantage of the second approach versus the first approach of PCA is that tSNE algorithm measures nonlinear effects in its clustering. To evaluate how representative this t-SNE approximation is to the real data, the Kullback-Leibler (KL) Divergence was used. This measurement ranges from 0 to infinite, with 0 meaning there is no difference between the t-SNE approximation and the underlying data.

FIG. 17 shows the tSNE analysis using M values after the following processing steps of removing all of the CpG sites with missing values in more than 75% of the samples, removing the CpG sites with no variance. tSNE analysis is devoid from scaling. A random state applied is 42 and a perplexity is 30.

The t-SNE plot in FIG. 17 visualizes the multidimensional methylation data processed from the skin samples, projecting multidimensional methylation data onto two dimensions to highlight patterns within the dataset. The crosses and circles distinguish between samples processed with targeted 4M CpG sequencing (from the melanoma sample from the forehead) and samples subjected to Whole-Genome Sequencing (em-seq). The t-SNE plot shows a distribution of the samples that suggests a degree of separability. The degree of separability is indicative of different ones of the methylation profiles or biological conditions. The t-SNE KL divergence score of approximately 0.917 suggests that the t-SNE plot may reasonably represent the high-dimensional structure of the data. This visualization aids in identifying inherent groupings and patterns in the methylation data.

The method enables constructing an epigenetic clock. The construction of the epigenetic clock involves a data preprocessing strategy. The chromosomes X, Y, and the mitochondrial DNA were excluded initially. The methylation values for the CpG sites for each sample with insufficient sequencing depth, namely less than 7 reads, were treated as missing values. The CpG sites and the samples with high levels of missing data were filtered out to ensure robustness, removing those CpG sites with missing values in more than 70% of the samples and the samples with more than 10% missing values. The samples with an average coverage below a certain threshold, namely 6 over Twist CpGs, were discarded. An average methylation signal for the remaining ones of the CpG sites was calculated across all available ones of the CpG sites within the regions covered by the probes of the panels, ensuring a representative and reliable signal of the methylation values for the constructing of the epigenetic clock. This method is termed a clustering-based approach.

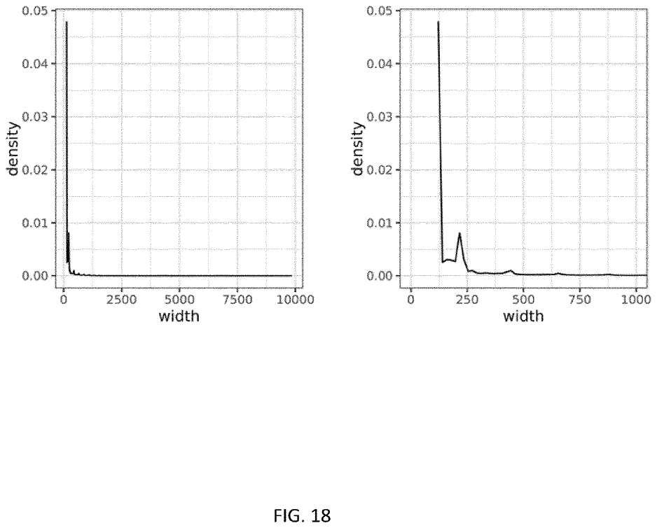

The clustering-based approach is supported by the observed fact that nearby CpG sites usually share similar CpG status. The exploitation of this piece of biological knowledge enables reducing the number of working features and removing co-correlated variables, a notorious problem when working with DNA Methylation data and that impacts the capacity of machine learning approaches. FIG. 18 illustrates the clustering-based approach. Although the panel explores approximately 4M CpGs, not all of these CpG sites are on their own, some of the CpG sites are extremely close-by. This file identifies which regions were targeted by Twist's probes, reducing the approximately 4M CpGs to ~550K regions defined by the boundaries set by Twist's Probes. In the following table, the characteristics of these regions are provided.

TABLE 1

| Min. | 1st Mean | Qu. Median | 3rd Qu. | Max. |
|---|---|---|---|---|
| 121.0 | 121.0 | 224.2 | 221.0 | 9842.0 |

FIG. 18 shows on the left side a density of region width. Peak at 121, second peak at 221. On the right side of FIG. 18, the same density of region is provided as on the left side, but zoomed to the region [0,1000].

TABLE 2
Quantiles of width

| | | | | | | | | | | | | | | | | | | |
|---|---|---|---|---|---|---|---|---|---|---|---|---|---|---|---|---|---|---|
| 0 | 5 | 10 | 15 | 20 | 25 | 30 | 35 | 40 | 45 | 50 | 55 | 60 | 65 | 70 | 75 | 80 | 85 | 90 |
| 121 | 121 | 121 | 121 | 121 | 121 | 121 | 121 | 121 | 121 | 121 | 121 | 149 | 175 | 204 | 221 | 221 | 269 | 433 |

Focusing on the last 10%

| | | | | | | | | | | |
|---|---|---|---|---|---|---|---|---|---|---|
| 90 | 91 | 92 | 93 | 94 | 95 | 96 | 97 | 98 | 99 | 100 |
| 433.00 | 440.00 | 478.00 | 554.00 | 628.88 | 672.00 | 798.00 | 891.00 | 1090.96 | 1368.98 | 9842.00 |

Only very few sites have over 1000 base pairs in width. Most of these regions have a span of ~121 base pairs.

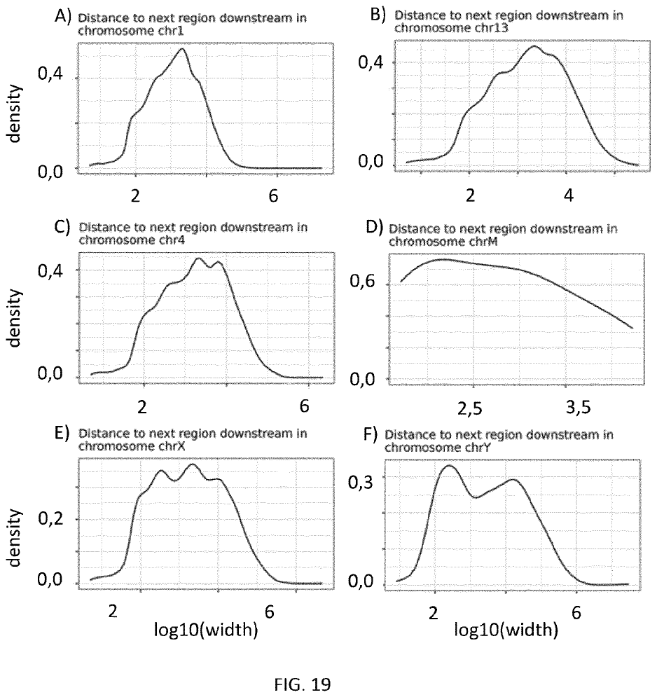

FIG. 19 shows how far apart the regions are from each other. This information is used because if, for example, regions were on average 8 base pairs apart from the next one of the regions, then it would be reasonable to assume that this division is not going to help reduce the effect of co-correlation in the resulting dataset, because CpG sites are used that are very close together. The information is obtained per chromosome. FIG. 19 illustrates, in particular, the density distribution of the log 10 distance between a region and the next downstream region per chromosome. As shown on FIG. 19, most chromosomes have a peak at ~3.2 (which corresponds to a distance of ~1600 bp), and smaller peaks at 4, 3 and 2 (Distance of 10000, 1000, 100 bp). The CpG sites within the span of the regions are defined in the regions file. In other words, the CpG file is probably derived from this main region-focused file. There are regions with no CpG sites, and precisely 3640 regions. These regions with no CpG sites are used for quality control purposes, to assess methylation in the non-CpG sites. The regions devoid of CpG sites comprise regions in any chromosome of the genome, including chr1 and chrY, but not chrM. An example region is chr1: 4071606-4071726, which has s the DNA sequence: GTTGCCATTAAACAAATATTGGACTCTCAGCATGACTCATGTTAGGATGC AGTTGTATCAAGGCAGCAGCTCTGCATAGGAAGTGTTGCTCTTGCATATT GCAGGGTATTAAGTGATTTTA. There are no CpG sites appearing in more than one region.

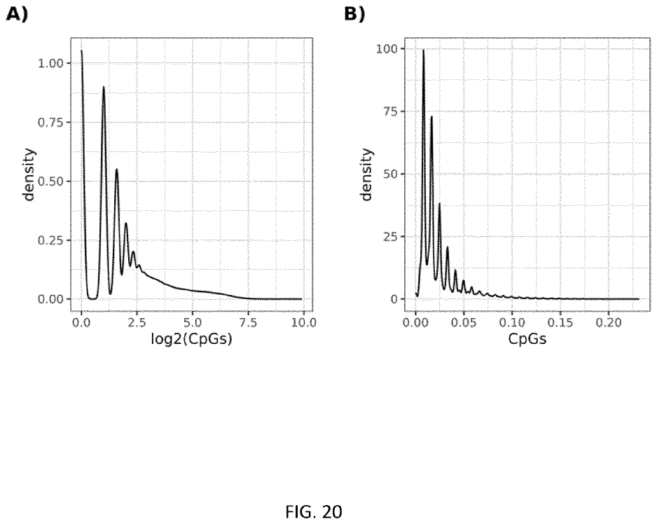

The log 2 uncorrected count of the CpG sites per region is shown on FIG. 20 (left side). FIG. 20 (left side) shows, in particular, the density distribution of the log 2 count of CpGs per region defined in the region probe. Most of the regions have only a single CpG site, closely followed by some of the regions with two CpG sites.

The right side of FIG. 20 shows terms of density (number of CpG sites divided by number of CpGs/n Base pairs). FIG. 20 (right side) shows, in particular, the density distribution of the number of the CpG sites per region when accounting for the length of the region itself. For most regions, the density of the CpG sites is similar and lies between 1-5% CpGs/base pairs. A value of 0.5 is the maximum and this value means that the whole region is a repeat of “CG.”

$${A}_{A} = 20 \qquad Equation 1$$

$$Y = \frac{A + 1}{{A}_{A} + 1}$$

$$if Y < 1 :$$

$$Age = log ( Y )$$

$$else :$$

$$Age = Y - 1$$

Equation 1 is an equation characteristic of a transformation. The value of the age is transformed using Horvath's function to account for younger individuals and use an output that better fits predictions using normal distributions. Since Horvarth's first clock, it has been observed that the epigenetic biomarkers of ageing do not follow the same methylation trend in individuals that are still in development compared to older individuals. To account for this trend, Horvath applied an invertible transformation of the age of individuals that has become commonplace in clock designs. Briefly, this transformation uses logarithmic values for individuals younger than an arbitrary age threshold over which individuals are considered adults. This transformation has a second benefit in that the transformation transforms the output of interest, i.e., age, from a positive-valued variable to allow negative values, which follows more closely the requirements to use Gaussian Distributions. The equation shows an age transformation applied to individuals. AA is the value considered to be adult age. This value is set to 20. A is the age of the individual to be transformed. The result of this transformation is further used as the variable to be predicted with the present model. This transformation is invertible, and the final results are shown after doing this reversal.

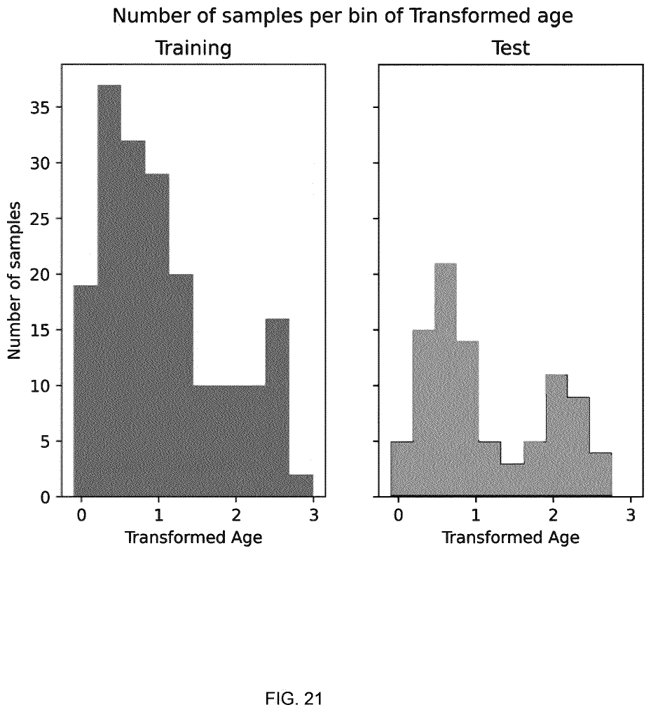

FIG. 21 shows a division of the data into training and test sets (67% and 33% respectively). The test set is used as a held-out test set, and the samples in the training set are used to train the model using Cross-Validation approaches. Using cross-validation enables reducing the chance of overfitting our model, making it more generalizable. More details about this approach are given in the following sections. FIG. 21 shows Train/test dataset distribution of Transformed Ages (The output of interest). Training set on the left, testing set on the right.

Although the data has already been reduced to a fraction of its original size whilst still maintaining its biological coherence, this dataset is still highly dimensional given the number of samples included in the dataset. It should be kept in mind about DNA methylation is that usually most regions will be, to some extent, associated with age, as the DNA methylation is a very broad output variable influenced by a multitude of confounders, and that although it is expected that expect most of the co-correlation, that is, the relative association between two features in terms of their methylation values, to have been dealt with thanks to the region coalescence method. Due to the inherent noise caused by the first problem, and to further avoid the possible effect of highly co-correlated regions, the number of regions input is reduced, so that the model only focuses on what is really important. To do so, an initial step of feature selection is performed in the training set. We highlight the importance of doing this after the training/test separation has been performed, to avoid data leakage and positively bias the results. This selection step is done calculating the Spearman Correlation between each region and the transformed age. As noted above, this Spearman Correlation metric measures the intensity and direction of association between two variables, with 0 meaning absolutely no association and −1 or 1 meaning a perfect relationship between the values in one variable and the other. The symbol indicates whether it is a negative or positive correlation, although in this case the negative correlation or the positive correlation is unimportant and only the absolute value of the association is recorded. This approach allows identification of associations without assuming the effect has to be linear. The Spearman Correlation is calculated between each region across the samples in training and age, and the distribution of values is explored. The regions are filtered using an arbitrary threshold of Spearman >0.30. This threshold reduces the number of regions from 530736 to only 14838 highly correlated regions. Feature selection is carried out in the training set before running the pipeline transformations. The selection is done through Spearman correlation between each region and its corresponding age. There are 530736 available regions in this dataset after applying the filtering steps (defined before).

The following distribution of values are obtained and disclosed in Table 3.

TABLE 3
corr.abs( ).quantile(np.linspace(0, 1, num = 11))

| | |
|---|---|
| 0.0 | 4.740271e−07 |
| 0.1 | 1.922742e−02 |
| 0.2 | 3.854074e−02 |
| 0.3 | 5.804421e−02 |
| 0.4 | 7.827975e−02 |
| 0.5 | 1.000850e−01 |
| 0.6 | 1.237448e−01 |
| 0.7 | 1.501605e−01 |
| 0.8 | 1.811259e−01 |
| 0.9 | 2.245211e−01 |
| 0.10 | 7.468888e−01 |

dtype: float64
corr [corr.abs( ) > 0.30

| | |
|---|---|
| X94 | −0.381802 |
| X119 | 0.308412 |
| X123 | −0.360120 |
| X175 | −0.379131 |
| X185 | 0.311380 |

. . .

| | |
|---|---|
| X537428 | 0.593928 |
| X537429 | 0.603844 |
| X537430 | 0.616029 |
| X537433 | −0.308506 |
| X537480 | 0.305945 |

Length: 14838, dtype: float 64

The top table of Table 3 shows a percentile distribution of the Spearman correlation between the value and transformed age. The bottom table of Table 3 shows a number of CpG sites with an abs (Spearman) over 0.30 (14838).

The CpG sites are filtered based on their Spearman correlation to transformed age, with an arbitrary minimum threshold of abs (0.30), reducing the number of initial regions to 14838.

Finally, after averaging the CpGs per region (n=530736 features) and the following spearman correlation filtering 14838 regions are defined to be used as the initial input regions for the machine learning model. To do this, we use the modules implemented by the Python package Scikit Learn (Link). This package provides multiple modules to produce machine learning models with high flexibility. The package also includes two classes that we leverage to easily tune the hyper-parameters of our model, as well as automatically implementing cross-validation.

The cross-validation is similar to the common train/test split also performed in this document. However, instead of not using the test data at all for training, the model is divided at random into multiple subgroups and, for each iteration, a different subgroup is used as the test set, and the remaining data goes through the normal step of the model. This approach allows 1) making sure that the data is not overfitted to the training test, because every time model is run, the input data is slightly different, 2) making sure the results observed are not the consequence of randomness and 3) ameliorating the effect of outliers in the data.

To leverage these capacities two types of objects implemented by Scikit-Learn are highlighted: Pipeline objects and GridSearchCV objects. The pipeline objects define which steps the inputted data should go through, finishing in an estimator object. In the current method, the pipeline is composed of the following steps:

Imputation of the missing values: the missing values are assigned the average value for that region. For this, the SimpleImputer class defined by Scikit-Learn is used.

Removal of the regions with low variance: Low variance implies low information to discern the output of interest. To further reduce the number of regions, the 15% least variable regions are filtered out using the object VarianceThreshold.

Scaling: Some methods require scaled values to work properly. As a default, Standard Normalization is performed with the object StandardScaler.

High correlation selection: Another step of selection by correlation is performed. In this case, the measurement used is Mutual Information. This metric determines how much information from one variable is communicated by another. In essence, this step is similar to the Spearman approach performed initially and is used as a sanity check.

PCA Decomposition: The remaining features after the previous filtering steps (9499) are transformed via PCA. As explained before, co-correlation is a frequent problem in the DNA methylation datasets, and it is particularly bad with certain machine learning approaches, like Lasso. The PCAs eliminate linear co-correlation from the data, as the PCA method combines the effect of multiple co-correlated dimensions into a single feature. It has been highlighted in the literature that the PCAs are good for DNAm clocks.

Lasso selection: Although the PCA strongly reduces the effect of co-correlation, some effects may still linger. Selection using Lasso Penalization is used for identifying those features and removing the effects of co-correlation, as well as keeping only the most necessary PCs for the output of interest.

Final estimator-Elastic Net: Finally, the selected PCs are used as features for the model itself. This model is based on the Elastic Net approach, which is a Generalized Linear Model with two penalization terms. This model is frequently observed with this type of complex datasets.

After the pipeline object is defined, the next main element is the GridSearchCV object. This class allows to easily specify certain hyperparameters that are to be tuned, including the type of scaling approach used, how many regions to use based on correlation, and others. Once those possibilities are defined, this object facilitates testing the model with every possible combination defined, as well as including Cross Validation. A 5-fold Cross validation per combination of hyperparameters is performed.

Following these steps results in the model observed in FIG. 8. The approach described in the image outlines the methodology used for developing an epigenetic clock based on DNA methylation data. After averaging the CpG sites per region (n=530736 features) and the following Spearman correlation filtering, which resulted in 14838 regions, a machine learning pipeline was constructed. This pipeline includes steps for imputation, feature selection, scaling, and dimensionality reduction through the PCA, and finally fitting using an Elastic Net model. The process also involved a hyperparameter optimization step using grid search to find the best parameters for the model and consistency assessment using cross validation.

Pipeline steps outline. A gridSearch was performed to identify the best choice for three parameters: (1) Choice of best scaling method, between Standard Scaling, Min Max scaling and no scaling at all; (2) Percentile of CpGs highly correlated to use, 10%, 30%, 50%, 70% or 90% (3) PCA.

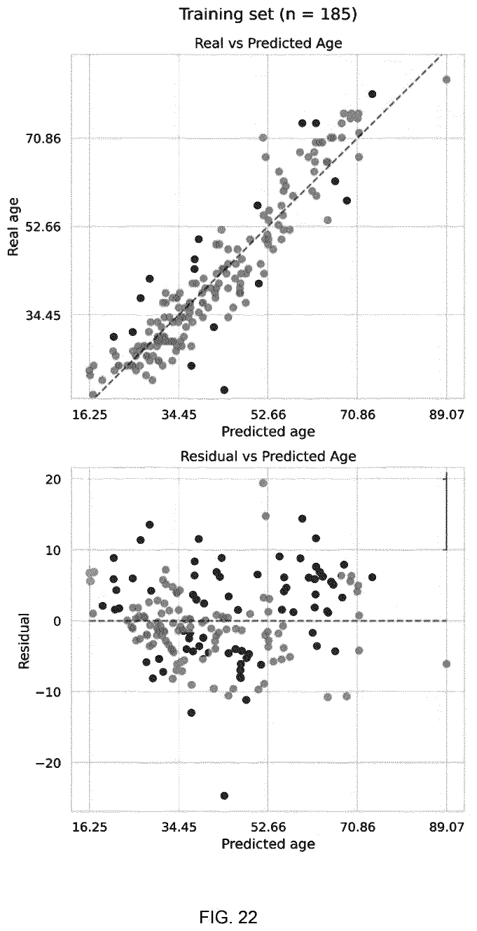

FIG. 22 shows prediction values in the training dataset. The top plot is the predicted value vs real value per sample, and the bottom plot is the distribution of residuals.

Table 4 shows the Mean Absolute Error (MAE), the Mean Squared Error (MSE), the Root Mean Squared Error (RMSE), the Median Absolute Deviation (MAD) and the squared correlation measurement (R2 score) of FIG. 22.

TABLE 4

| | |
|---|---|
| The Mean Absolute Error (MAE) is: | 4.43 |
| The Mean Squared Error (MSE) is: | 33.22 |
| The Root Mean Squared Error (RMSE) is: | 5.76 |
| The Median Absolute Deviation (MAD) is: | 3.91 |
| The R2 score is: | 0.87 |

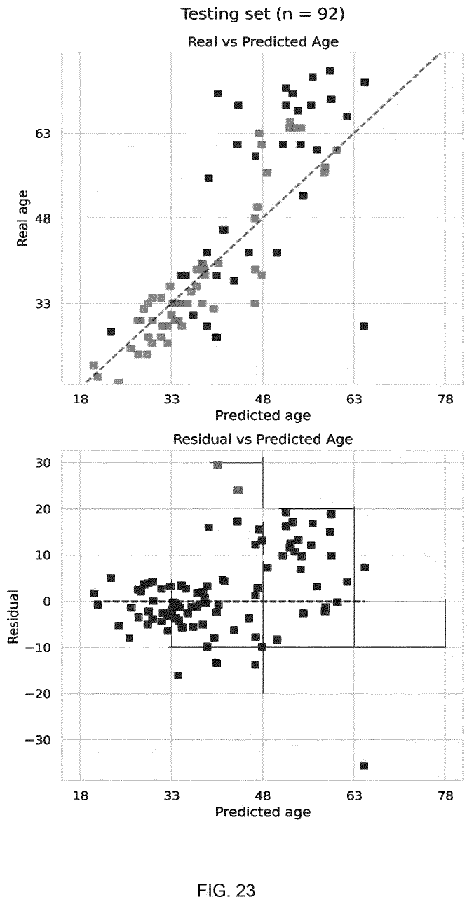

FIG. 23 shows prediction values in the testing dataset. The top plot is the predicted value vs real value per sample, and the bottom plot is the distribution of residuals. In terms of the Elastic Net model, the best parameters found were |1_ratio of 0.1 and alpha of substantially 0.26.

Table 5 shows the MAE, the MSE, the RMSE, the MAD and the R2 score of FIG. 23.

TABLE 5

| | |
|---|---|
| The Mean Absolute Error (MAE) is: | 7.0 |
| The Mean Squared Error (MSE) is: | 93.74 |
| The Root Mean Squared Error (RMSE) is: | 9.68 |
| The Median Absolute Deviation (MAD) is: | 4.26 |
| The R2 score is: | 0.65 |

This model is further validated with targeted sequenced samples from the Twist panel that were produced after the development of the model.

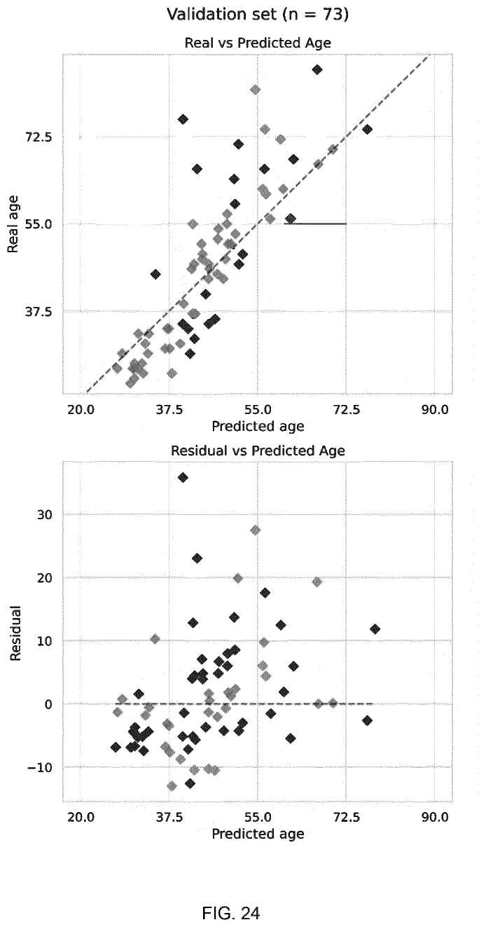

FIG. 24 shows prediction using MitraClock v3.0.0 on new samples from the targeted sequencing project (n=73).

Table 6 shows the MAE, the MSE, the RMSE, the MAD and the R2 score of FIG. 24.

TABLE 6

| | |
|---|---|
| The Mean Absolute Error (MAE) is: | 6.84 |
| The Mean Squared Error (MSE) is: | 87.64 |
| The Root Mean Squared Error (RMSE) is: | 9.36 |
| The Median Absolute Deviation (MAD) is: | 5.11 |
| The R2 score is: | 0.7 |

As observed in FIG. 24?, the resulting metrics are extremely similar to what is observed in the testing set, highlighting the consistency of the model to be applied in other not explored datasets. These samples were not used for the initial filtering either, which also indicates that there is no data leakage in the model biassing the results.

A small feasibility study was designed to assess the epigenetic changes on the skin with topical use of 0.025% tretinoin. The study was conducted over 2 months (56 days) and collected samples from baseline and post-product application at day 14, day 28 and day 56 post product application. CB, SK, and DK were also sampled 1 and 2 weeks prior to the experiment, accounting for the control data points (Ctrl). This dataset was used as a validation set for the model training and building as described above.

The ages of the participants are described in the table 7 below:

TABLE 7

| Initials of participant | Age | Avg. Predicted age |
|---|---|---|
| CB | 30 | 31.68 |
| DK | 31 | 32.05 |
| AA | 31 | 34.22 |
| PP | 33 | 33.49 |
| NN | 40 | 39.85 |
| SK | 35 | 36.26 |
| JT | 32 | 30.72 |
| V125_t2_A | 50 | 49.25 |

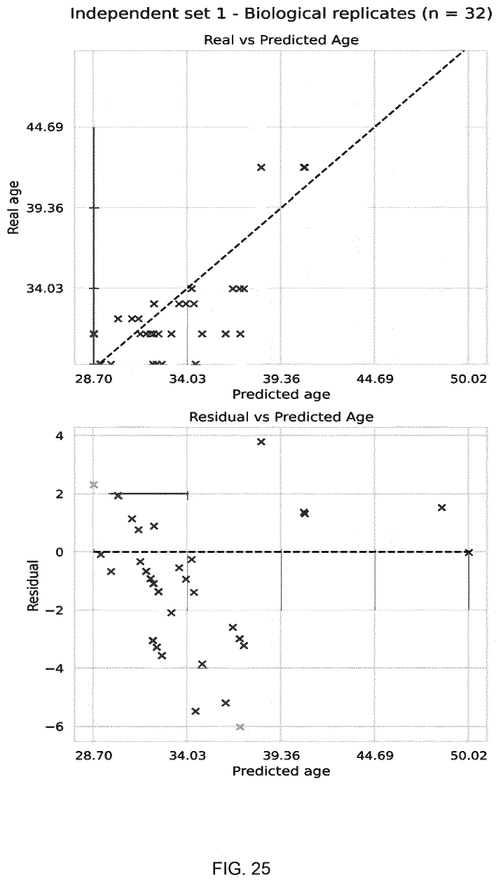

The result in validation dataset is as observed in FIG. 25.

Table 8 shows the MAE, the MSE, the RMSE, the MAD and the R2 score of FIG. 25.

TABLE 8

| | |
|---|---|
| The Mean Absolute Error (MAE) is: | 2.02 |
| The Mean Squared Error (MSE) is: | 6.6 |
| The Root Mean Squared Error (RMSE) is: | 2.57 |
| The Median Absolute Deviation (MAD) is: | 1.38 |
| The R2 score is: | 0.78 |

The error measurements observed in each of the 4 datasets are summarized in table 9 below. Table 9 illustrates in particular the number of samples and error metrics of the 4 datasets. Table 9 comprises the MAE, the MSE, RMSE, the MADR, and the R2 score of the 4 datasets.

TABLE 9

| | Training | Testing | | Independent |
|---|---|---|---|---|
| Dataset | set | set | Validation | set |
| n samples | 185 | 92 | 73 | 32 |
| MAE | 4.43 | 7 | 6.84 | 2.02 |
| MSE | 33.22 | 93.74 | 87.64 | 6.6 |
| RMSE | 5.76 | 9.68 | 9.36 | 2.57 |
| MAD | 3.91 | 4.26 | 5.11 | 1.38 |
| R2 score | 0.87 | 0.65 | 0.7 | 0.78 |

### REFERENCE NUMERALS

10 subject
20 keratinocyte sample
30 methylated nucleic acid sequences
40 machine-learning model
60 training dataset
70 system
80 input device
90 memory
100 processor
110 validation dataset

1. A method for determining a stage of an epigenetic property of an epidermis of a subject, comprising:
providing a keratinocyte sample obtained non-invasively from the subject;
identifying methylated nucleic acid sequences in the keratinocyte sample by enzymatic methyl sequencing (EM-seq); and
using a trained machine-learning model to process the identified methylated nucleic acid sequences and thereby to determine the stage of the epigenetic property.

2. The method of claim 1, wherein the methylated nucleic acid sequences are methylated deoxyribonucleic acid (DNA).

3. The method of claim 1, wherein the epigenetic property is melanoma.

4. The method of claim 1, wherein the epigenetic property is skin ageing.

5. The method of claim 1, wherein the trained machine-learning algorithm is an elastic net regression model.

6. A computer-implemented method for training a machine-learning model for determining a stage of an epigenetic property of a subject, the method comprising:
receiving a training dataset comprising a plurality of data elements, wherein the data elements comprise methylated nucleic acid sequences, identified by enzymatic methyl sequencing, associated epidermal properties, and epidermal stage results; and
training the machine-learning model using the training dataset.

7. The method of claim 6, wherein the methylated nucleic acid sequences originate from training samples.

8. The method of claim 6, wherein the trained machine-learning model is an elastic net regression model.

9. The method of claim 6, wherein the trained machine-learning model uses averaged out signal of methylation values from CPG sites in region.

10. A system for determining a stage of an epigenetic property of a subject, the system comprising:
an input device for inputting at least one methylated nucleic acid sequence identified by enzymatic methyl sequencing;
a memory for storing a trained machine learning model; and
a processor for using the inputted at least one methylated nucleic sequence and the stored trained machine learning model to determine the stage of the epigenetic property.

11. The system of claim 10, wherein the methylated nucleic acid sequences originate from training samples.

12. The system of claim 10, wherein the trained machine learning model is an elastic net regression model.

13. Use of the method of claim 1 for determining at least one of the stage of melanoma or skin age in the subject.

14. The method of claim 1, wherein the sample is obtained using an adhesive tape.
